(19) United States
(12) Patent Application Publication
(10) Pub. No.: US 2005/0182312 A1
(43) Pub. Date: Aug. 18, 2005

(54) Contact tonometer using MEMS technology

(75) Inventors: John C. Bruce, Jacksonville, FL (US); Michael J. Crocetta JR., Jacksonville, FL (US)

Correspondence Address: MEDTRONIC, INC.;Attn: Patent Department, 710 Medtronic Parkway, Minneapolis, MN, 55432, US

(73) Assignee: Medtronic Xomed, Inc.

(21) Appl. No.: 10/778,006

(22) Filed: Feb. 12, 2004

(51) Int. Cl. A61B 3/16; A61B 5/00

(52) U.S. Cl. 600/399; 600/405; 600/406

(57) ABSTRACT

An contact tonometer for sensing intra-ocular pressure (IOP) including a micro-electro-mechanical system (MEMS) device forming a transducer/sensor at or in contact with a contact end of the tonometer where the cornea is contacted and electronics receiving an electrical signal from the transducer and processing the signal to produce a display indicative of intra-ocular pressure.

### BACKGROUND OF THE INVENTION

1. Field of the Invention

The present invention relates to the use of micro-electro-mechanical systems (“MEMS”) technology in the fabrication of pressure or force sensing monitors for the human body in the medical field and, more particularly, for sensing intra-ocular pressure (IOP).

2. Brief Discussion of the Related Art

The eye is one of the most important organs of the human body. It is hard to imagine how difficult and lonely it would be if you lost visual contact with the colorful world. Cataracts, glaucoma and age-related macular degeneration are the three major diseases of the eye that rob older people of vision. Among these three, glaucoma is the leading cause of blindness, accounting for 12 percent of new cases of blindness each year in the United States. Glaucoma is often called the “silent thief” because most people who develop glaucoma cannot feel it until it is too late to be mitigated by medical treatment.

The most significant indicator of glaucoma has been found to be elevated inner eye pressure. This elevated eye pressure damages the optic nerve and can deteriorate into total blindness for the patient. Accordingly, early detection is crucial for successful glaucoma treatment, and elevated inner eye pressure, or intra-ocular pressure (“IOP”), is the signature of glaucoma. The measurement of IOP has been the most effective diagnostic tool for early detection of glaucoma. Actual IOP can only be obtained through a direct method, such as inserting a cannula connected to a manometer into the anterior and posterior segments of the eye. It is obvious that this method cannot be used in routine eye examinations. Thus, IOP is measured non-invasively, which is defined as tonometry, during routine eye exams.

During the past century several tonometry methods have been established and applied in clinical practice. The following is a brief history of tonometry methods:
Early 1900: Schiotz tonometer
1950s: Goldmann tonometer
1960s: MacKay-Marg tonometer
1970s: Non-contact tonometers
1980s: Handheld tonometers such as the Tono-Pen® applantation tonometer marketed by Medtronic Xomed, Inc., and portable Goldmann tonometers such as the Draeger or Perkins devices.

In the past, Tonometry was classified in general as two types according to the method of corneal distortion. They are:
applanation tonometry—a small portion of cornea is flattened, and
indentation tonometry—a small portion of cornea is “indented.”
Indentation tonometry, represented primarily by the Schiotz tonometer, was the dominant method to measure IOP during the first half of the last century. It gradually faded away after the Goldmann Applanation Tonometer (“GAT”) was invented. The disadvantages of the Schiotz tonometer were:
   patient apprehension,
   anesthesia required,
   good patient cooperation needed,
   corneal abrasions possible,
   required a physician (not staff members), and
   significant aqueous displacement.

Applanation tonometry can be divided into two categories
Variable force (constant area) contact applanation tonometry (GAT, MacKay-Marg tonometer (“MMAT”), Draeger tonometer, Perkins tonometer, and Tono-Pen® applantation tonometer, and
Constant force (variable area) applanation tonometry (non-contact).
   The major IOP measurement method currently is variable force applanation contact tonometry.

The (IOP) is constantly above the atmospheric pressure to preserve the shape of the eyeball, thereby ensuring a stable alignment of the optical components. There are two separate compartments inside the eye: the aqueous cavity and the vitreous cavity. The front compartment (aqueous cavity) is filled with a fluid called aqueous. Within the aqueous cavity are two areas: the anterior chamber (in front of the iris) and the posterior chamber (behind the iris). The vitreous cavity is filled with a jellylike substance called vitreous.

While the vitreous is relatively inert and stable, the aqueous humour serves to provide the metabolic oxygen demands of the lens and a portion of the cornea, both without blood vessels. Additionally, the vitreous plays a key role in maintaining IOP by balancing formation and drainage rates of aqueous humour and to act, in the anterior chamber, as a component of the optical system.

Aqueous is produced by the ciliary bodies and pumped into the posterior chamber, where it circulates through the papillary space and into the anterior chamber. It drains out of the anterior chamber through the trabecular meshwork and reaches Schlemm's canal. It is then transported through a network of aqueous veins and gradually absorbed into the blood supply by vessels in the conjunctiva. If the flow of aqueous is impeded along its route, IOP will rise which can damage the optic nerve.

Statistical mean IOP is 16 mmHg with a standard deviation of 3 mmHg. Although there is no clear line between safe and unsafe IOP, it is commonly called elevated IOP when the IOP exceeds 21 mmHg. Many factors can affect the IOP, such as time of the day, heartbeat, respiration, exercise, fluid intake, systemic medications, topical drugs, cannabis and alcohol (transient decrease), caffeine (transient increase), recumbent position (higher), aging (higher), and genetics.

There are two basic forms of glaucoma, closed-angle glaucoma and open-angle glaucoma. Closed-angle glaucoma occurs where the root of the iris blocks the route where aqueous flows into the trabecular meshwork. Open-angle glaucoma occurs where the trabecular meshwork is clogged. Open-angle glaucoma is the more common form. Beyond these two, it has been observed that some patients developed glaucoma despite having normal IOP (low tension glaucoma). The cause of this type of glaucoma is still unknown.

The Imbert-Fick law is the foundation of all types of applanation tonometers. It was introduced late in the last century and then applied to IOP measurement. However, it was widely accepted and became the dominant tonometry method only in the 1950s after the invention of the Goldmann applanation tonometer. In accordance with the Imbert-Fick law, if an infinitely thin, perfectly flexible, perfectly elastic, and dry spherical container with internal pressure P is flattened (applanated) by an external force W, the flattened area A, external force W and internal pressure Patient have the following relationship:
Pₜ=W/A.

The human eye does not satisfy all of the conditions required in the Imbert-Fick law in that the cornea is about 0.5 mm (mean value) thick rather than infinitely thin, the cornea tissue is not perfectly flexible and a small portion of the deforming force is balanced with the tension rather than IOP force. The cornea has limited rigidity rather than being perfectly elastic, the cornea is wet and the surface tension of the tear film tends to pull the applanating surface onto the cornea.

In 1957, Goldmann and Schmidt found through their experiments that the Imbert-Fick law could be more realistically presented in the IOP measurement by the equation where
W+s=Pₜ×Aᵢ+b
where
W=external deforming force;
s=extra force due to surface tension tending to pull the applanating surface against the cornea;
Pₜ=IOP;
Aᵢ=flattened area of cornea; and
b=force to bend the cornea.

Goldmann and Schmidt also found that s and b are balanced if the diameter of the applanation area is 3.06 mm. Therefore, the equation becomes
W=Pₜ×π×(3.06 mm)²/4=7.35Pₜ
or
Pₜ=W/7.25g/mm²=10W mmHg.

Goldmann and Schmidt noted that the measurements are only reliable in eyes with normal human corneas. These equations are not valid in eyes of examined animals because the corneas of the animals' eyes are different from the human corneas.

The Goldmann Applanation Tonometer (GAT) was designed based on the experimental data from average thickness and rigidity of human corneas. When a cornea is thicker than average, the reading of GAT is higher than true IOP; when a cornea is thinner, the reading is lower. When the cornea is weakened, either by excimer ablation or by stromal edema the readings of GAT are always lower.

As mentioned above, the force to bend the cornea and the force due to surface tension cannot be neglected in the IOP measurement of human eyes. In 1959, Mackay and Marg invented a special applanation tip (“MMAT”) where those forces are physically eliminated; therefore, the Imbert-Fick law can be directly applied to the IOP measurement. Unlike other tonometers, the Mackay-Marg applanation tip is formed of two areas. A central area (about 1-2 mm in diameter) is the sensing area. A force or pressure sensor is implemented there. The central sensing area is surrounded by a guarding area (about 3 mm in diameter).

This type of design has the advantages of the force to bend the cornea does not affect the IOP measurement since bending is done by the guarding area. The surface tension of tears does not affect the IOP measurement since it happens in the conjunction of the guarding area and eyeball, there not being a need to carefully monitor the applanation to reach total flatness as in the case of GAT since only the central area is used to calculate the IOP, the tip being coverable by a disposable rubber membrane to reduce possibility of infection and also to protect both the tip and the eye. The central sensing part of the MMAT can be a plunger combined with a force sensor. It has been found through animal (rabbit) testing that there is an ideal initial plunger extension. For a 1.5-mm diameter plunger, a 5 micron initial projection is ideal.

During MMAT experiments, it has been observed that the MMAT tonograms share a common interesting format in rising sharply to the first crest dipping to a first trough, rising again slowly to a central maximum, dipping to a second trough, rising again to a second crest and then falling sharply to the baseline. This format is explained as the corneal bending effect during IOP measurement by MMAT: first crest, representing bending of the cornea at the limit of the applanated area, the first trough, representing balance of applanation force and IOP force, the central maximum representing raised pressure resulting from the applanation, and the depth of the trough representing a measure of corneal stiffness.

Findings from experiments with MMAT reveal that crests and troughs are prominent when the diameter of the transducer tip is under 2 mm and almost smoothed to a plateau by 3 mm diameter, special precautions must be taken to place the tonometer squarely on the cornea to get correct IOP readings, ordinary 75 micron thick rubber films tend to degrade the crest by reducing sensitivity so that use of a thinner covering or none at all may be desirable for this purpose; and corneal bending rather than corneal buckling is responsible for the crest and trough of the curve.

A particularly effective and easy-to-use applanation tonometer is the Tono-Pen® applantation tonometer marketed by Medtronic Xomed, Inc. (Opthalmic division) and described in U.S. Pat. No. 4,747,296 to Feldon et al. The Tono-Pen® applantation tonometer is a portable, hand held instrument utilizing micro strain gauge technology with a 1.5 mm transducer tip. As described in the Feldon et al patent, the Tono-Pen® applantation tonometer has an elongate housing mounting an activation switch, batteries, a display, electronic circuitry and a microprocessor mounted on a printed circuit board and a strain gauge sensor/transducer. In use, the Tono-Pen® applantation tonometer is moved to contact the cornea and displays the average of four independent readings, along with a statistical coefficient, with accuracy comparable to the GAT.

Some of the areas where the Tono-Pen® and other applanation tonometers may be improved include reduced weight to facilitate use, the permitting of a more gentle contact with the cornea, ruggedness, such that the applantation tonometer can more easily withstand being dropped without permanent damage, ease of manufacture at bulk rates, repeatability of manufacture of the transducer/sensor as well as simplified manufacture of the applantation tonometer.

MEMS are miniature micro-electro-mechanical systems, sometimes referred to as miniature electromechanical components formed of micromachined transducers in silicon, primarily, and often integrated with electronic microcircuits, herein referred to as “electronics.” The transducers can be sensors and/or actuators based on electrostriction, electromagnetic, thermoelastic, piezoelectric, piezoresistive, capacitive, acoustic, strain gauge, differential pressure and/or optical effects. MEMS fabrication uses techniques previously used for microelectronics permitting accurate and bulk microfabrication such that MEMS devices provide enhanced performance and are resistant to failure due to corrosion and wear.

MEMS devices have been contemplated for use in the past for placement within the eye or on a contact lens to sense IOP due to their small size; however, the unexpected advantages of the use of MEMS technology in both hand held and table mounted tonometers have not been recognized nor has there been any recognition of the manner in which MEMS technology can be used to improve tonometers.

### SUMMARY OF THE INVENTION

The present invention is generally characterized in a contact tonometer for sensing intra-ocular pressure (IOP) of an eye comprising a contact surface for making contact with a surface of said eye; a micro-electro-mechanical system (MEMS) device connected to said contact surface wherein said MEMS device produces an electrical signal corresponding to the force applied by said contact surface to said surface of said eye when said surface of said eye is contacted by said contact surface; an electronics unit for receiving said electrical signal and converting said electrical signal to an IOP signal that is representative of the IOP of the eye; a display for receiving the IOP signal from the electronics unit and displaying information that is representative of the IOP of the eye; and a power source for supplying electrical power to said electronics unit and said display. In one embodiment, the contact tonometer is a hand held device. In another embodiment, the contact tonometer further comprises a first housing member capable of being attached to a human finger for containing the contact surface and the MEMS device. This first housing member may also contain the electronics, the display and the power source, or any combination thereof. In yet another embodiment, the contact tonometer further comprises a second housing member coupled to said first housing member and capable of being attached to a human hand for containing the display. This second housing member may also contain the electronics, the activation switch and the power source, or any combination thereof.

### BRIEF DESCRIPTION OF THE DRAWINGS

FIG. 1 is a schematic diagram of the contact tonometer of the present invention.

FIG. 2 is a perspective of acontact tonometer according to one embodiment of the present invention.

FIG. 3 is a side view, partly in section, of one embodiment of the contact tonometer of the present invention.

FIG. 4 is a side view, partly in section, of another embodiment of the contact tonometer of the present invention.

FIG. 5 is a side schematic view of an embodiment of the contact tonometer sensor of the present invention.

FIG. 6 is a side schematic view of another embodiment of the contact tonometer sensor of the present invention.

FIG. 7 is a side schematic view of another embodiment of the contact tonometer sensor of the present invention.

contactcontact FIG. 8a is a side schematic view of an embodiment of the contact tonometer sensor of the present invention.

FIG. 8b is a side schematic view of an embodiment of the contact tonometer sensor of the present invention when force is applied.

FIG. 9a is a side schematic view of an embodiment of the contact tonometer sensor of the present invention.

FIG. 9b is a side schematic view of an embodiment of the contact tonometer sensor of the present invention when force is applied.

FIG. 10a is a side schematic view of an embodiment of the contact tonometer sensor of the present invention.

FIG. 10b is a side schematic view of an embodiment of the contact tonometer sensor of the present invention when force is applied.

FIG. 11 is a schematic diagram of the distribution of charge in a piezoelectric material.

FIG. 12 is a schematic diagram of an embodiment of the contact tonometer sensor of the present invention.

FIG. 13 is a schematic diagram of an embodiment of the contact tonometer sensor of the present invention.

FIG. 14 is a schematic diagram of the cylinder driver and pickup of the embodiment of FIG. 13.

FIG. 15 is an electronic schematic diagram of the electronic circuit of the cylinder driver and pickup of the embodiment of FIG. 13.

FIG. 16 is a schematic diagram of an embodiment of the contact tonometer sensor of the present invention.

FIG. 17a is a schematic diagram of an embodiment of the contact tonometer sensor of the present invention.

FIG. 17b is a schematic diagram of the embodiment of the contact tonometer sensor of the present of FIG. 17a in a stressed condition.

FIG. 18 is a schematic diagram of one embodiment of the contact tonometer sensor of the present invention.

FIG. 19 is a schematic diagram of an embodiment of the contact tonometer sensor of the present invention of FIG. 18.

FIG. 20 is a perspective of a contact tonometer according to one embodiment of the present invention.

### DESCRIPTION OF THE PREFERRED EMBODIMENTS

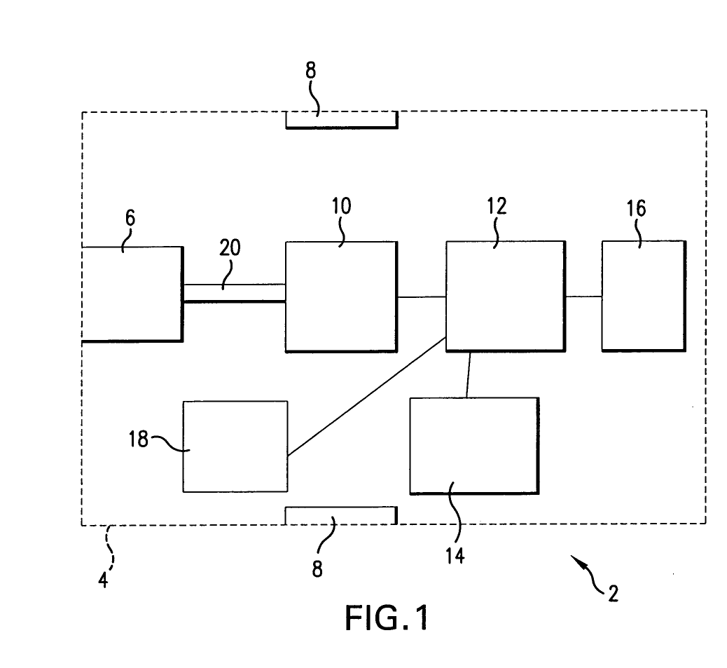

As shown schematically in FIG. 1, contact tonometer 2 according to the invention includes a housing 4 having a distal or contact end 6 and, in a preferred embodiment, a gripping portion 8 proximal to the contact end 6. The contact tonometer 2 includes a MEMS device 10 acting as a transducer to measure the force applied by the contact end 6 to the patient's cornea and to produce an electrical signal representative thereof. The contact tonometer 2 includes electronics and/or microprocessor (“electronics”) 12, a source of power 14 and a display 16. In the preferred embodiment, the electronics 12, source of power 14 and display 16 are integral parts of the housing 4. However, in another embodiment, they may be separate from housing 4.

Electronics 12 processes electrical signals from the MEMS device 10 and supplies a signal to display 16 causing display 16 to display information representative of the determined IOP. The source of power 14 is connected to the electronics 12 and display 16 and provides power to the electronics 12 and display 16. An activation switch 18 is preferably disposed on the housing 4 and is connected to electronics 12. Activation switch 18 allows a user to activate the electronics 12. However, in another embodiment activation switch 18 may be separate from housing 4.

Source of power 14 is preferably a battery. However, the source of power 14 could also be any other source of power such as common household electrical power provided through a power line. Where the source of power 14 is common household electrical power, the contact tonometer 2 will need to be connected to the source of common household power through a power cable (not shown) connected to such source of common household power as is well understood in the art. Additionally, where the source of power is common household power, the source of power 14 may include a power supply to provide an appropriate voltage to the electronics 12 and display 16. Where the source of power 14 is a battery, the battery may be, but is not required to be, mounted in the housing 4 in a manner to provide balance for the contact tonometer 2 as required.

Although a specific arrangement of and connection between the MEMS device 10, electronics 12, source of power 14, display 16 and activation switch 18 has been described, it is clear that other configurations and connections may be used so long as the functionality of the components separately and combined is maintained. The following examples are given for the purpose of illustration and are not intended to limit the possible combinations and configurations that will be clear to those skilled in the art. For example, activation switch 18 could be electrically located between the source of power 14 and the electronics 12 to control power being provided to electronics 12. Additionally, the MEMS device 10, electronics 12 and display 16 could all be connected by a bus that allows power and information to pass between the devices. Further, all or some of these devices could be formed together in an integrated device such as an integrated circuit (IC).

Electronics 12 includes any suitable electronics to take the signal sent from the MEMS device 10 and operate on such signal according to an algorithm such as that described above in connection with the Goldmann and Schmidt equation described above to correlate the force measured by the MEMS device 10 to IOP. Electronics 12 preferably includes a microprocessor but may include application specific integrated circuits (ASIC) or hard-wired electronics. The signal processing described in the Feldon et al '296 patent and used in the Tono-Pen® contact tonometer is representative of one embodiment of the electronics 12 for use with the contact tonometer of the present invention.

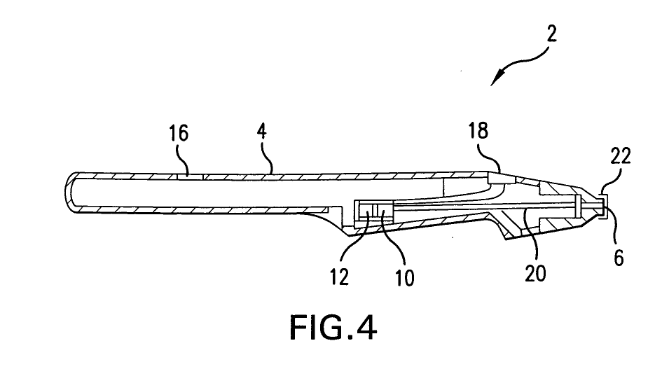

The MEMS device 10 is connected to the contact end 6 through a connection member 20. Connection member 20 in one embodiment (FIG. 3) is direct contact between the MEMS device 10 and the contact end 6. In another embodiment (FIG. 4), connection member 20 is a rigid arm connecting contact end 6 to MEMS device 10. Preferably, a membrane 22 (FIGS. 3 and 4) is disposed at the contact end 6 to be positioned between the contact end 6 and the cornea of a patient's eye. This membrane 22 is preferably disposable, non-reactive and bio-compatible with the cornea and therefore provides a clean, sanitary surface for contact with the eye with each use.

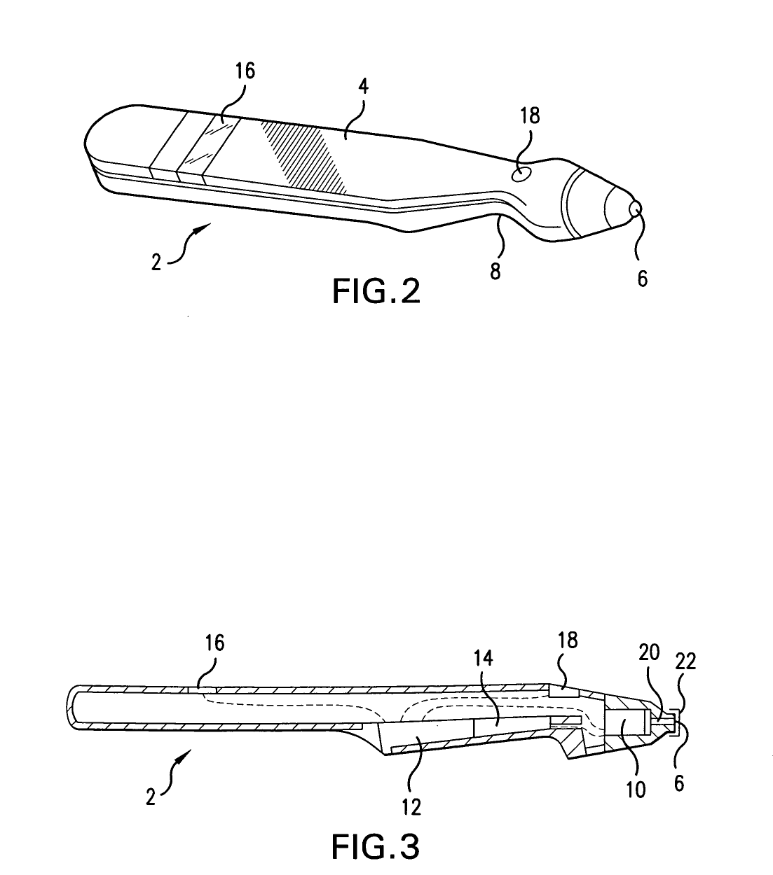

In one embodiment shown in FIG. 2, the housing and consequently appearance of the contact tonometer 2 of the present invention is essentially the same as that shown in U.S. Pat. No. 4,747,296 to Feldon et al which is incorporated herein by reference. However, since the appearance of the contact tonometer 2 is determined largely by the housing 4, housing 4 may take many forms. The function of housing 4 is to provide a platform for the contact end 6, to house the MEMS device 10 and the electronics 12, in one embodiment to provide a platform for the display 16 and, where the contact tonometer 2 is hand held, to allow the contact tonometer 2 to be gripped and handled by a user. Consequently, housing 4 may take many forms and shapes so long as these functions are accomplished. The MEMS device 10 senses the force applied to the patient's cornea by the contact end 6 of the device and creates an electric signal related thereto. The MEMS device 10 can be a micro-mechanical device such as those incorporating moving members such as deflecting micro-cantilevers, deflecting diaphragms and other micro-machined devices such as are known to the micro-mechanical art. The MEMS device 10 can also incorporate a moving gas or fluid as is well understood in micro-fluidic or micro-pneumatic devices. Additionally, the MEMS device 10 may also incorporate at least one of electrostatic, magnetic, piezoelectric, electromagnetic, inertial, pneumatic, hydraulic or thermal micro-actuation mechanisms. This MEMS technology has already been applied particularly in micro-mechanical switches and sensors and is therefore well known in the art.

As a result, the specific structure associated with such devices is not critical to the invention. However, the ability of such MEMS devices 10 to detect the force applied to the contact end 6 and to produce an electrical signal representative of such force is critical to the invention.

One advantage of using MEMS devices in place of macro-devices or discrete devices in an contact tonometer 2 is that MEMS devices may be fabricated in large numbers through processes analogous to those used in the production of semiconductors. Typically, MEMS devices 10 are produced as packaged chips. These “chip-packages” are usually typical IC-Chip packages made from ceramic, plastic, metal, etc. The fact that such MEMS devices 10 are employed in connection with an contact tonometer 2 and the corresponding performance, cost, packaging and reliability advantages is a key to the invention.

In one embodiment shown in FIG. 3, the contact tonometer 2 has a MEMS device 10 mounted adjacent to the contact end 6 so that connection member 20 is essentially direct contact between contact end 6 and the MEMS device 10. In this embodiment, the MEMS device 10 is preferably disposed at or near the contact end 6.

In another embodiment shown in FIG. 4, the contact tonometer 2 has a MEMS device 10 mounted proximally away from but mechanically connected to the contact end 6 by the connection member 20. In this embodiment, the mechanical connection may be accomplished through the connection member 20 where connection member 20 is any connection between the contact end 6 and the MEMS device 10 that transfers the force applied to the contact end 6 to the MEMS device 10. One example of such a connection is a rigid arm attached between the contact end 6 and the MEMS device 10. In this embodiment, the MEMS device 10 may be located anywhere within the housing 4 so long as the MEMS device 10 is physically separated from the contact end 6. Here, the connection member 20 transfers motion of the contact end 6 to the MEMS device wherever located.

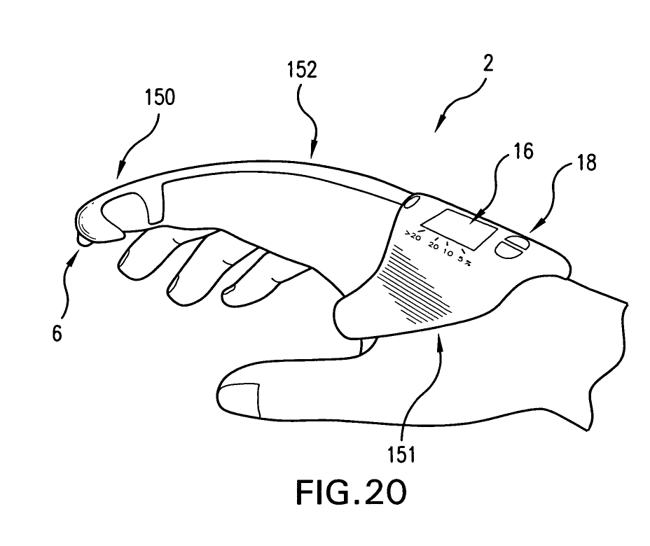

In yet another embodiment shown in FIG. 20, the contact tonometer 2 has a housing consisting of a first member 150 wherein the contact end 6 and the MEMS device 10 (FIG. 1) are housed connected to a second member 151 wherein the electronics 12 (FIG. 1), the power source (14 FIG. 1), activation switch 18 and the display 16 are housed. The connector 152 between first member 150 and second member 151 may be a rigid or preferably flexible. The connector is capable of transmitting electrical signals from the MEMS device 10 to the electronics 12. In this embodiment the first member 150 is capable of being attached to the end of a human finger and the second member 151 is capable of being attached to a human hand. However in other embodiments, the electronics 12, the activation switch 18 and display 16 or any combination thereof may also be housed in the first member 150. In yet other embodiments wherein the connector 152 is flexible, the second member 151 may be mounted to a table or placed anywhere that is convenient for the operator of the contact tonometer 2.

The MEMS device 10 acts as a transducer or sensor and is formed with MEMS technology preferably using bulk or etched manufacturing processes to produce a transducer or sensor of a suitable type. In any of the embodiments of the MEMS device 10 shown, the MEMS device 10 actually measures the force applied to the MEMS device 10 to the patient's cornea and produces an electrical signal corresponding to such force. Processing of such electrical signal, such as through the application of an algorithm operating on the electronics 12 produces a signal representative of the IOP.

In one embodiment, the electronics 12 for producing transducer or sensor signals from the MEMS device 10 are fabricated in the MEMS device 10 as is conventional for integrated circuits. As a result, in this embodiment the MEMS device 10/electronics 12 combination need only be electrically connected to the source of power 14 and display 16 as described above.

### MECHANICAL

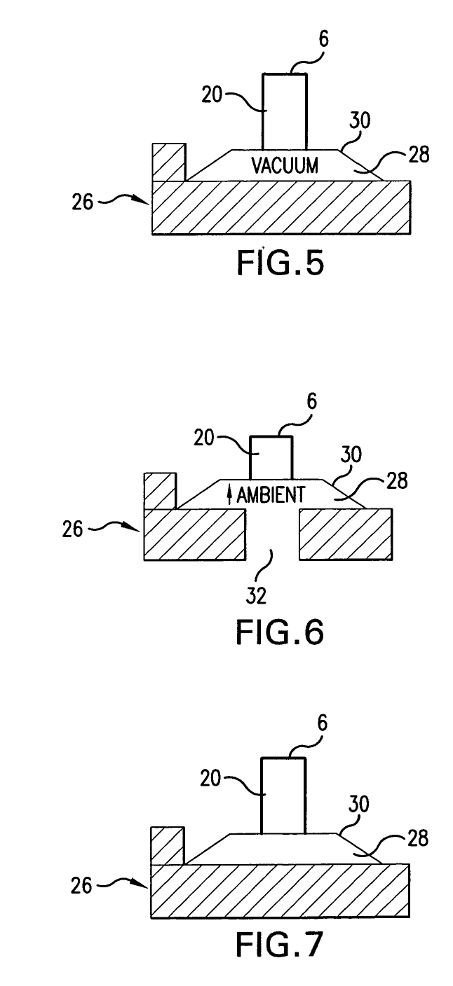

In a simple form, the MEMS device 10 can be photoetched resistors in a Wheatstone bridge arrangement in a substrate 26 as is well understood in the art. An absolute pressure measurement arrangement of such a MEMS device 10 is illustrated in FIG. 5 wherein an internal chamber 28 is located below a movable membrane or diaphragm 30. Internal chamber 28 is sealed and held under vacuum. Diaphragm 30 is connected to contact end 6 in any manner described above. When no force is applied to diaphragm 30, the substrate 26 is in an equilibrium condition. In this state, the resistors in the Wheatstone bridge will have a certain resistance which will be the baseline resistance of the MEMS device 10. When a force is applied to contact end 6 and consequently to diaphragm 30, diaphragm 30 interacts with substrate 26 to place a different stress on the resistors of the Wheatstone bridge. As a result, the resistance of the Wheatstone bridge will change. This change is proportional to the force applied to diaphragm 30 from the contact end 6. Consequently, the force applied by the contact end 6 on the patient's cornea will produce a change in the resistance of the Wheatstone bridge in a way that is highly correlated to such force.

A gauge pressure measurement arrangement of MEMS device 10 is illustrated in FIG. 6 wherein the substrate 26, internal chamber 28 and diaphragm 30 have been modified to provide a passage 32 connecting internal chamber 28 to ambient atmospheric pressure. The operation of this MEMS device 10 is as described above in connection with the embodiment shown in FIG. 5. In this embodiment, the difficulty of maintaining a vacuum within the internal chamber 28 is eliminated. However, in the unstressed condition, the MEMS device 10 must be allowed to come to an equilibrium condition before an IOP measurement can be taken. This equilibrium condition is dependent, in part, on the ambient atmospheric pressure which is constantly changing. However, the change in ambient pressure is so small over the time span needed to take an IOP measurement that once the MEMS device 10 has reached an equilibrium condition and the electronics 12 is calibrated to indicate “zero” pressure, subsequent IOP measurements will be highly accurate.

A sealed gauge pressure measurement arrangement of MEMS device 10 is illustrated in FIG. 7 wherein the substrate 26, internal chamber 28 and diaphragm 30 are as described in connection with the embodiment of FIG. 5 with the exception that instead of a vacuum within internal chamber 28, a fixed common reference pressure is placed within internal chamber 28. This embodiment eliminates the difficulty of providing a vacuum within the internal chamber 28 and also eliminates the need to allow the MEMS device 10 to equilibrate before each use to accommodate the changing atmospheric pressure.

### PIEZOELECTRIC

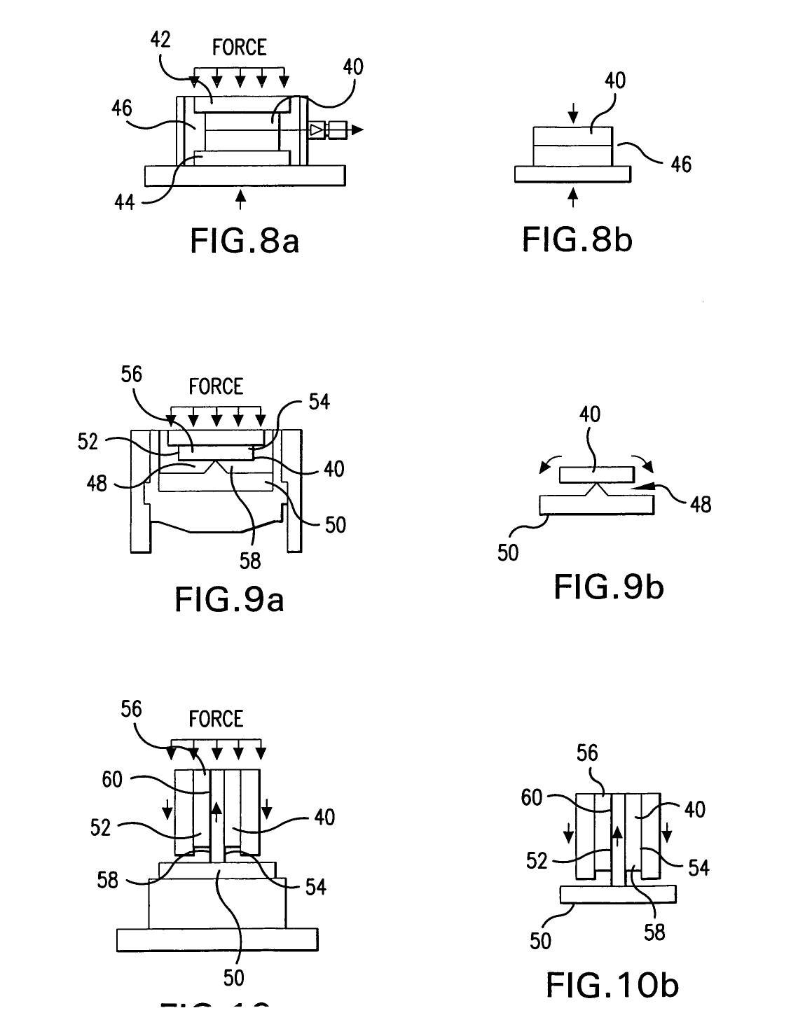

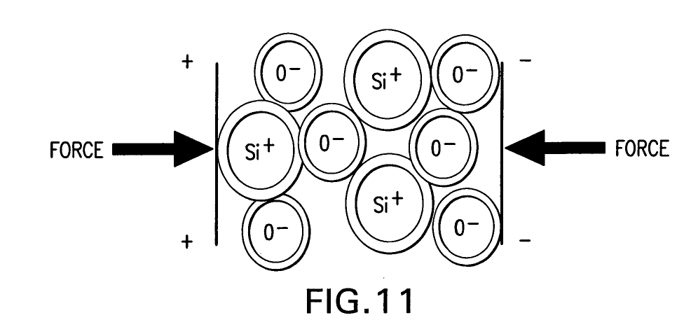

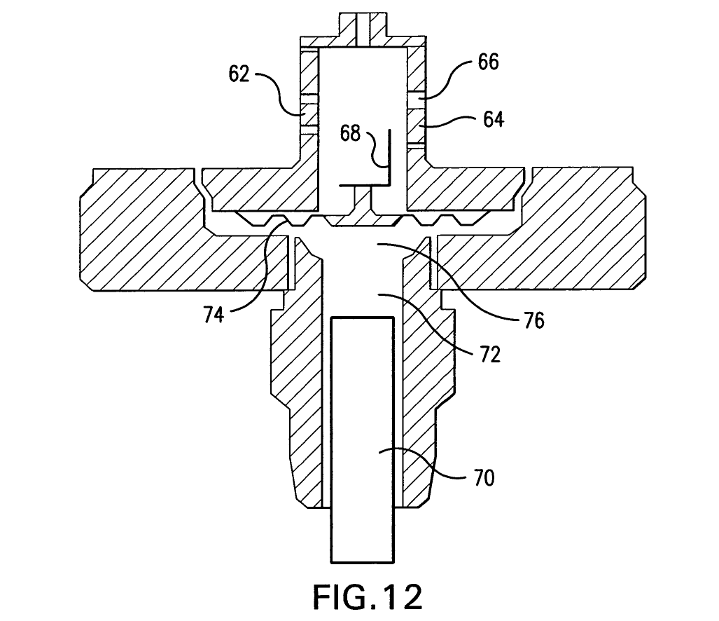

An example of a MEMS device 10 using a piezoelectric transducer for use with the contact tonometer 2 is shown in FIGS. 8-10 and described in detail below. Where the MEMS device 10 is a piezoelectric arrangement, when the piezoelectric elements are strained by an external force, displaced electrical charge accumulates on opposing surfaces. When piezoelectric elements are strained by an external force, displaced electrical charge accumulates on opposing surfaces. FIG. 11 schematically shows the displacement of electrical charge due to the deflection of the lattice in a naturally piezoelectric quartz crystal. The larger circles having the notation “Si⁺” represent silicon atoms while the smaller circles having the notation “O⁻” represent oxygen. As shown in FIG. 11, when a force is applied to the crystal, charge of opposite polarity accumulates on opposite sides of the crystal.

Many different sizes and shapes of piezoelectric materials can be used in piezoelectric sensors for MEMS device 10. Acting as true precision springs, different element configurations, such as compression, flexural and shear, offer various advantages and disadvantages, flexural being preferred for the present invention. With stiffness values on the order of 15E6 psi (104E9 N/m2), which is similar to that of many metals, piezoelectric materials produce a high output with very little strain. In other words, piezoelectric sensing elements have essentially no deflection and are often referred to as solid-state devices. For this reason, piezoelectric sensors are rugged and feature excellent linearity over a wide amplitude range. Crystalline quartz, either in its natural or high-quality, reprocessed form, is one of the most sensitive and stable piezoelectric materials available and is the preferred material for a MEMS device 10 in this embodiment.

Piezoelectric materials can only measure dynamic or changing events. Piezoelectric sensors are not able to measure a continuous static event as would be the case with measuring inertial guidance, barometric pressure or weight. As a result, the electronics associated with the piezoelectric MEMS device 10 of this embodiment must be able to detect the change of status of the MEMS device 10. While static events will cause an initial output due to a change from the previous condition to the current condition, this signal will slowly decay or drain away based on the piezoelectric material or attached electronics time constant. This time constant corresponds with a first order low pass filter and is based on the capacitance and resistance of the device. This low pass filter ultimately determines the low frequency cut-off or measuring limit of the device. In the preferred embodiment of the invention, the MEMS device 10 should have a time constant that is about equal to the time it takes for the clinician to tap the patient's cornea with the contact tonometer 2. Also, the cutoff frequency should be high enough to allow complete measurement of the IOP.

An example of a highly sophisticated MEMS device 10 using a piezoelectric transducer for use with the contact tonometer 2 is shown in FIGS. 8-10 wherein both shear and normal forces can be measured. The MEMS device 10 in these embodiments could be fashioned after that described in Kane, B. J., et. al., “Force-Sensing Microprobe for Precise Stimulation of Mechanosensitive Tissues,” IEEE Transactions on Biomedical Engineering, vol. 42, no. 8, August 1995, pp. 745-750, which is incorporated herein by reference. The embodiments of FIGS. 8-10 provide an extremely accurate means to measure IOP by assuring normality of the transducer surface to the eye.

In the embodiment of the invention shown in FIGS. 8a, 9a and 10a, the MEMS device 10 is a piezoelectric device. Acting as true precision springs, the different element configurations shown in FIGS. 8-10 offer various advantages and disadvantages. In FIGS. 8-10 schematic diagrams labeled FIGS. 8b, 9b and 10b represent in shaded area the piezoelectric crystals while the arrows indicate how the piezoelectric crystal material is being stressed.

In the embodiment shown in FIG. 8, the MEMS device 10 includes a piezoelectric crystal 40. The crystal 40 is placed between and in contact with a first face 42 and a second face 44. Crystal 40 has a central slot 46 that runs essentially parallel to both first face 42 and second face 44. First face 42 is either in direct contact with the contact end 6 or is in mechanical contact with contact end 6 through a connection member 20 as described above. In this way, force of the contact tonometer 2 contacting the patient's eye is directed to contact end 6 and then either directly or indirectly to the first face 42. Second face 44 is anchored to the contact tonometer 2 so that it provides a steady base for crystal 40. When a force is applied to the contact end 6, and therefore also applied to the first face 42, the force is applied to the crystal 40. In response to the application of the force, crystal 40 will attempt to move into more firm contact with the second face 44. However, because second face 44 is anchored to the contact tonomter 20, second face 44 will resist movement due to the force applied to crystal 40. As a result, crystal 40 will be stressed and charge will accumulate on opposite sides of the central slot 46 (FIG. 8b). The accumulated charge is collected on opposite sides of the central slot 46 and, when connected to electronics 12, produces a signal representative of the force applied to the crystal 40 which, in turn, corresponds to the force applied to the contact end 6 which in turn corresponds to the patient's IOP.

In the embodiment shown in FIG. 9, the MEMS device 10 also includes a piezoelectric crystal 40. The crystal 40 is placed over a pivot point 48 on a base 50. Crystal 40 in this embodiment has a first side 52 and a second side 54 on either side of the pivot point 48 and a top 56 and a bottom 58. Either or both first side 52 or second side 54 is in either direct contact with the contact end 6 on the top 56 or is in mechanical contact with contact end 6 through a connection member 20 as described above connected to the contact end 6 on one end and the top 56 on the other end. In this way, force of the contact tonometer 2 contacting the patient's eye is directed to contact end 6 and then either directly or indirectly to either or both of first side 52 or second side 54. Base 50 is anchored to the contact tonometer 2 so that it provides a steady base for pivot point 48 where pivot point 48 contacts the bottom 58 of crystal 40. When a force is applied to the contact end 6, and therefore also applied to the first side 52, second side 54 or both, contact between the bottom 58 of crystal 40 and the pivot point 48 prevents the part of crystal 40 between first side 52 and second side 54 from moving. As a result, crystal 40 flexes around the pivot point 48. As crystal 40 is stressed on either side of the pivot point 48, opposite charge accumulates on the top 56 and bottom 58 of crystal 40 (FIG. 9b). The accumulated charge is collected from top 54 and bottom 58 and, when connected to electronics 12, produces a signal representative of the force applied to the crystal 40 which, in turn, corresponds to the force applied to the contact end 6 which in turn corresponds to the patient's IOP.

In the embodiment shown in FIG. 10, the MEMS device 10 again includes a piezoelectric crystal 40. In this embodiment, crystal 40 is mounted on a central cylinder 60 that is rigidly attached to a base 50 and extends through crystal 40. Crystal 40 in this embodiment also has a first side 52 and a second side 54 on either side of the central cylinder 60 and a top 56. Top 56 is in either direct contact with the contact end 6 or is in mechanical contact with the contact end 6 as described above. First side 52 and second side 54 are in direct contact with the top 56. Base 50 is anchored to the contact tonometer 2 so that it provides a steady and relatively immovable base for central cylinder 60. Central cylinder 60 supports crystal 40 and acts as a pivot point for crystal 40 as torque is applied to crystal 40 through force applied from the contact end 6 to the top 56.

In this way, force of the contact tonometer 2 contacting the patient's cornea is directed to contact end 6 and then either directly or indirectly to either or both of first side 52 or second side 54 which in turn causes crystal 40 to be flexed or torqued around central cylinder 60. But, because central cylinder 60 is relatively immovable, crystal cannot move but instead is compressed on either first side 52, second side 54 or both. This compression causes charge to be distributed on opposite faces of crystal 40. As crystal 40 is stressed around central cylinder 60, opposite charge accumulates on the top 56 and bottom 58 of crystal 40 (FIG. 10b). The accumulated charge is collected from top 56 and bottom 58 and, when connected to electronics 12, produces a signal representative of the force applied to the crystal 40 which, in turn, corresponds to the force applied to the contact end 6 which in turn corresponds to the patient's IOP. The advantage of this embodiment is that it offers a well-balanced blend of low sensitivity to base strain and low sensitivity to thermal inputs.

### OPTICAL

A MEMS device 10 using optical pressure transducer/sensor arrangements, such as that shown in FIG. 12, can be used with the contact tonometer 2 to detect the effects of minute motions due to changes in pressure and generate a corresponding electronic output signal to pass to electronics 12. A source diode 62 is used as a light source that projects light toward a measuring diode 64 and a reference diode 66. Source diode 62 may be a light emitting diode (LED) that emits visual or infrared light. A vane 68 is attached to contact end 6 and moves as contact end 6 moves in contact with the patient's cornea. Vane 68 may be either connected directly to contact end 6 as described above or, as shown in FIG. 12, contact end 6 may be a piston 70 that is placed in a bore 72. In this embodiment, vane 68 is located on a diaphragm 74. A chamber 76 is formed between diaphragm 74 and bore 72 that is filled with a fluid. As piston 70 moves in response to the force applied by the contact tonometer 2 on a patient's cornea, pressure builds within the chamber 76. This pressure causes the diaphragm 74 to deflect with in turn causes the vane to move in the light path of light source 62. As vane 68 moves with movement of contact end 6, in either the embodiment of direct contact with contact end 6 or in the embodiment shown in FIG. 12, vane 68 blocks more and more of the light from source diode 62 as it is directed toward the measuring diode 64 and thus changes the amount of light received by measuring diode 64.

This optical MEMS device 10 embodiment may also compensate for aging of the source diode 62 through the use of by means of the reference diode 66. Reference diode 66 is located so that it is never blocked from receiving light from the source diode 62 by the vane 68. Because the reference diode 66 is never blocked by the vane 68, any degradation of the signal received by the reference diode 66 will be due to deterioration, such as by the build-up of dirt or other coating materials on the optical surfaces or aging of the source diode 62. Consequently, the signal produced by the source diode 62 may be used as a baseline to which the signal produced by the measuring diode 64 can be compared.

The optical MEMS device 10 embodiment is relatively immune to temperature effects because the source diode 62, measurement diode 64 and reference diode 66 are affected equally by changes in temperature. Moreover, because the amount of movement of the contact end 6 required to make measurements is very small (typically under 0.5 mm), hysteresis and repeatability errors are nearly zero. An optical MEMS device 10 such as described herein also does not require much maintenance, has excellent stability and is designed for long-duration measurements and are available with ranges from 5 psig to 60,000 psig (35 kPa to 413 MPa) and with 0.1% full scale accuracy.

### RESONANT/VIBRATION

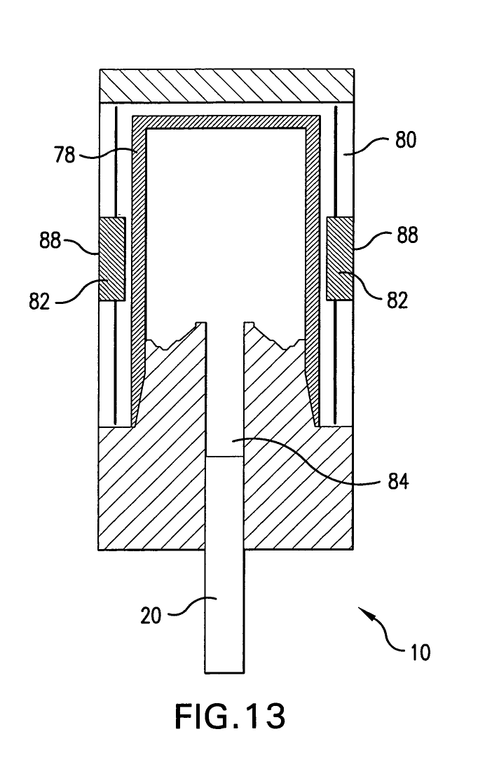

MEMS device 10 can be of the resonant/vibration type. In such a MEMS device 10, a structure is caused to resonate at its natural frequency and this frequency is modulated as a function of the input parameter, in this case the force applied to the patient's cornea. A MEMS device 10 according to this embodiment is shown in FIG. 13. MEMS device 10 in this embodiment includes resonant cylinder 78 preferably made of a flexible metallic bellows, an outer cylinder 80 that surrounds the resonant cylinder 78, a cylinder driver and pickup 82 and an input channel 84.

As stated above, resonant cylinder 78 may be made of a metallic bellows. The flexible metallic bellows of resonant cylinder 78 is used to modulate the force applied to the MEMS device 10 as a function of the pressure applied to MEMS device 10 through contact with the contact end 6 and the patient's cornea. It is preferable to use high-elasticity, low-creep and low hysteretic materials in the fabrication of the resonant cylinder 78. This results in a highly stable and high-resolution measurement method. Resonant cylinder 78 is either made of a ferromagnetic material or has pieces of ferromagnetic material placed in or on it to allow it to be driven at a resonance frequency as will be described hereafter.

Preferably, a vacuum is placed between the resonant cylinder 78 and the outer cylinder 80. This vacuum separates resonant cylinder 78 from outer cylinder 80 to decouple movement of resonant cylinder 78 from the outer cylinder 80. The vacuum here is preferably a high-quality internal vacuum around the resonant cylinder 78 thereby eliminating the viscous damping effects that an internal gas environment would present to the resonating resonant cylinder 78 and to reduce the drive power requirements as will be explained hereafter in connection with the cylinder driver and pickup 82. This internal vacuum also prevents ideal gas thermal expansion forces that would act upon the resonant cylinder 78 and the large variable effects that airborne moisture would cause.

The interior of the resonant cylinder 78 and the input channel 84 is preferably filled with fluid but could also be filled with a gas. The input channel 84 is connected to the connection member 20. Connection member 20 in this embodiment is fashioned so that a portion of connection member 20 extends into the input channel 84 and acts as a piston. As a result, as the connection member 20 is moved as a result of contact between the connection member 20 and the patient's cornea, the portion of connection member 20 in input channel 84 interacting with the fluid within input channel 84 causes the pressure of the fluid within the resonant cylinder 78 to increase.

In this embodiment of the MEMS device 10, the resonant cylinder 78 is caused to oscillate at its resonance frequency by the “driver” portion of the cylinder driver and pickup 82. The resonance frequency is the frequency at which maximum mechanical output (vibration) occurs with a minimum energy input. For this reason, the total energy required to cause the resonant cylinder 78 to vibrate at its resonance frequency is small. The resonance frequency is therefore the frequency of motion at which maximum efficiency results for vibration of the resonant cylinder 78. Changes in the resonant frequency will occur due to the different pressures induced within the resonant cylinder 78 as a result of contact between the connection member 20 and a patient's cornea as described above.

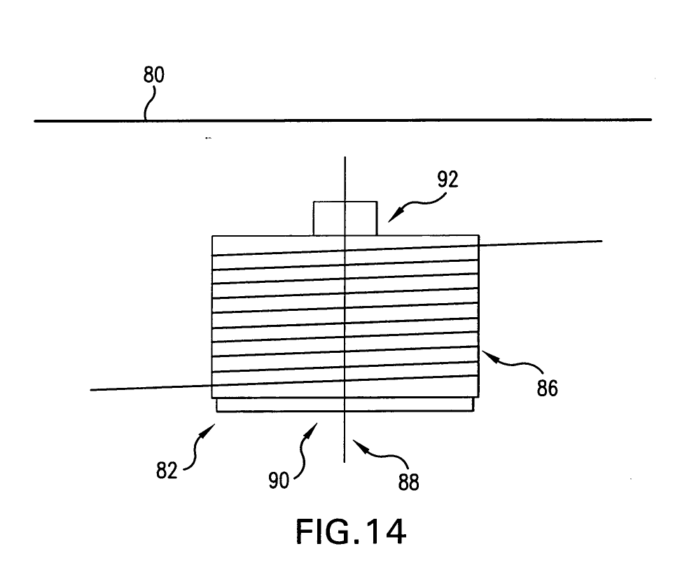

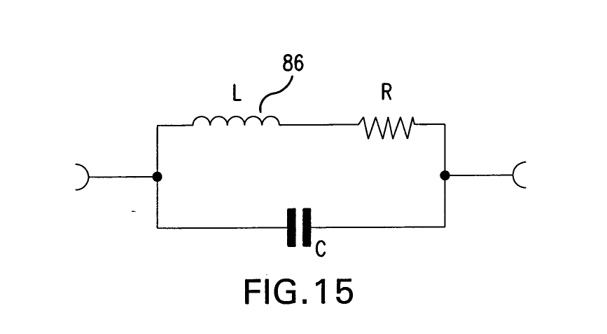

The cylinder driver and pickup 82 performs the double function of both causing the resonant cylinder 78 to vibrate and also sensing the vibration of resonant cylinder 78. This is preferably accomplished by either electromagnetic or piezoelectric methods in an analogous method to electric guitar pickups. Here, as shown in FIG. 14, at least one coil 86 of insulated wire is placed near the surface of resonant cylinder 78 opposite where the connection member 20 contacts the input channel 84. The coil 86 has a central axis 88 around which the coil 86 is formed. The central axis 88 is oriented perpendicular to the outer surface of resonant cylinder 78. In a preferred embodiment, a permanent magnet 90 is placed through the coil 86 so that a pole 92 of the magnet 90 extends away from the coil 86. In alternate embodiments, the magnet 90 may be placed below coil 86 with a soft iron core placed within the coil 86. Also, it may be desirable to be able to move the magnet 90 closer to or away from the surface of the resonant cylinder 78 to “tune” the cylinder driver and pickup 82. Also, it may be desirable to surround the coils with some sort of an electromagnetic shield such as a metal case or isolating tape.

Direct current is passed through the coil 86 thereby creating a magnetic field with lines of magnetic flux passing through the center of coil 86. This magnetic field interacts either directly with the material of resonant cylinder 78 if this material is ferromagnetic or with the piece or pieces of ferromagnetic material placed on or in the material making up the resonant cylinder 78. By varying the electric current passed through the coil 86, the pull on the resonant cylinder 78 is varied. By pulsing the application of electric current through the coil 86 the resonant cylinder 78 can be made to vibrate. When the application of current through the coil 86 coincides with the resonant frequency of the resonant cylinder 78, the amplitude of the vibration of resonant cylinder 78 will be the largest.

The “pickup” portion of cylinder driver and pickup 82 also senses the movement of the resonant cylinder 78 as resonant cylinder 78 vibrates in response to the application of electric current to coil 86 as described above. The movement of the ferromagnetic material of resonant cylinder 78 in the magnetic field of the permanent magnet causes the magnetic flux through the coil 86 to change. Since the coil 86 is a good conductor, the change in magnetic flux is opposed in the coil 86 by the induction of an alternating current. The change in magnetic field that is created from the AC current is opposite to that of the change in magnetic field in the coil 86 due to a principle known as Lenz's Law. The reason for the induction of an alternating current in the coil 86 rather than a direct current is due to the motion of the of the vibrating resonant cylinder 78 as the surface of the resonant cylinder moves both towards and away from the pole 92 of the pickup in the same way that the voltage of an AC current increases and decreases.

As the surface of resonant cylinder 78 moves closer to the pole 92, the magnetic flux within coil 86 increases while the magnetic flux in coil 86 decreases while the surface of the resonant cylinder 78 moves farther away from the pole 92. The magnetic field lines flow through the coil 86 and a portion of the surface of resonant cylinder 78. With the surface of resonant cylinder 78 at rest, the magnetic flux through the coil 86 is constant. But, as coil 86 is activated to magnetically couple with resonant cylinder 78, the flux changes. This change of flux induces an electric voltage in the coil 86. This vibrating resonant cylinder 78 induces an alternating voltage at the frequency of vibration, where the voltage is proportional to the velocity of the motion of the surface of resonant cylinder, not the amplitude of such vibration. Furthermore, the voltage depends on the material, thickness and magnetic permeability of the resonant cylinder 78 and the strength of the magnetic field created by coil 86 and the distance between the magnetic pole 92 and the resonant cylinder 78.

From an electrical standpoint, the pickup portion of the cylinder driver and pickup 82 is shown in FIG. 1518. The windings of coil 86 have an inductance L in series with an resistance R and is parallel to both a winding capacitance C. Of these electrical components, by far the most important quantity is the inductance which it depends on the number of windings, the magnetic material in the coil and the geometry of the coil 86. Although present, the resistance doesn't have much influence and for practical purposes can be neglected. As described above, when the resonant cylinder 78 is vibrating, an AC voltage is induced in the coil 86. The capacitance C is the sum of the winding capacitance of the coil and the capacitance of the wiring connecting coil 86 to the electronics that powers the coil 86 and processes the information sensed by coil 86. The resonant frequency of coil 86 depends on both the inductance L and the capacitance C.

Although the cylinder driver and pickup 82 described above has been described with a single coil 86, such single coils are sensitive to magnetic fields generated by transformers, fluorescent lamps, and other sources of interference and are prone to pick up hum and noise from these sources. Therefore, it is preferably that instead of a single coil for coil 86, dual coils that are electrically out of phase, such as those used in “humbucking” pickups for guitars, are used to minimize this interference. Because these coils for coil 86 are electrically out of phase, common-mode signals (i.e. signals such as hum that radiate into both coils with equal amplitude) cancel each other.

The “driver” and “pickup” of cylinder driver and pickup 82 are connected in a closed-loop system whereby the “driver” portion of cylinder driver and pickup 82 can be driven in response to the sensed vibration of the metallic resonant cylinder 78 by the “pickup” portion of the cylinder driver and pickup 82. Because this is a closed loop system, the frequency that the “driver” drives the resonant cylinder 78 can be adjusted to the frequency requiring the minimum energy to drive the resonant cylinder 78. This minimum energy is found at its most mechanically-efficient frequency or “maximum-Q” response point. This frequency is the resonant frequency for resonant cylinder 78.

Counter circuitry then counts the oscillator output over some defined time-averaging window. Such circuitry as is well known in the quartz watch industry can be used to detect the resonant frequency. The frequency response of the resonant sensor is therefore a direct function of the number of time-averaged samples provided per second and is generally low. Alternatively, the frequency of the resonant structure can be measured utilizing a period measurement system to provide a much wider measurement bandwidth. Period measurement systems rely upon a second internal time base operating at a much higher frequency than the resonant structure to provide adequate period resolution.

### FLUID

MEMS device 10 can be of the fluidic type. In such devices, a force, such as that applied by the contact end 6 as it contacts a patient's cornea, is applied to a gas or fluid contained in a chamber. The force is transmitted through the gas or fluid to a moving member. The moving member of such MEMS devices 10 can move in response thereto as, for example, by distortion, deformation, translation, deflection, rotation, torsion or other motion. This motion is then detected by, for example, a strain gauge to produce the electrical signal representative of the force applied.

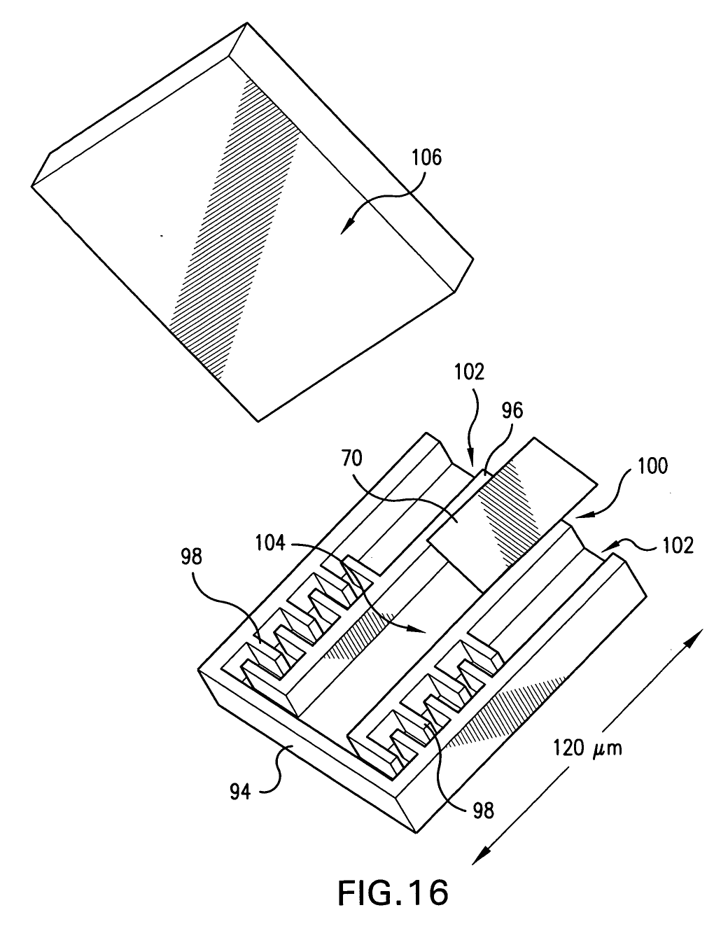

In such a MEMS device 10, shown in FIG. 16, a substrate 94 having a top surface 96 has a series of microfluidic channels 98 micro-machined, formed or cut in its top surface 96. The channels 98 have a central inlet 100 and two outlets 102. The channels 98 function as a pressure drop/pulse attenuator for fluid flow through the MEMS device 10. Central inlet 100 forms a chamber 104 near the outermost edge of substrate 94. A piston 70 such as is described in connection with the embodiment of FIG. 13 is placed in a fluid-tight position in chamber 104 and is connected to the contact end 6 either directly or through the connecting member 20. In this way, movement of the contact end 6 causes the piston 70 to move within the chamber 104. The chamber 104 is filled with a fluid. As the contact end 6 moves in response to the force applied by the applanantion tonometer 2 to the patient's cornea, the piston 70 is moved into the chamber 104 causing an increase in fluid pressure inside the chamber 104. The fluid flows from the chamber 104 through the central inlet 100, through the channels 98 and out through the two flanking outlets 102 at a reduced pressure. Since the flow rate is directly related to the pressure applied to the contact end 6, measuring the flow rate by any of the commonly known methods for measuring the flow rate provides a direct correlation to the pressure applied to the contact end 6 and thus to the patient's IOP.

As an example of the size of the MEMS device 10 in this embodiment, the MEMS device 10 is approximately 100×120 um, with a channel depth of 10 um. The MEMS device 10 also includes a lid 106 to seal the top of the MEMS device 10. This lid 106 is made by sealing another chip of corresponding dimensions to the substrate onto the top surface 96 of the substrate 94 thus making a so-called “flip chip package”.

### CAPACITIVE

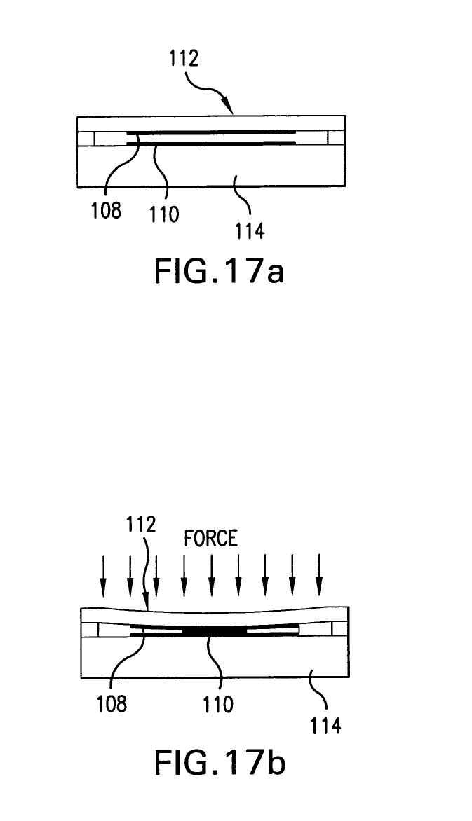

MEMS device 10 may also be a capacitive device. Such a MEMS device 10 is shown in FIG. 17. The MEMS device 10 of FIG. 17 includes a first plate 108 and a second plate 110 that are parallel to each other and form a capacitor. The first plate 108 is fixed to a ceramic diaphragm 112 that is in contact, either directly or through connection member 20, with the contact end 6. Diaphragm 112 flexes in response to force changes applied to the contact end 6. The second plate 110 is attached, with a rigid glass seal, to a ceramic substrate 114 that is insensitive to pressure changes. As the force applied to the contact end 6 varies (FIG. 17b), the diaphragm 112 flexes and the distance between the first plate 108 and second plate 110 changes. This MEMS device 10 thus produces a variable capacitor that is highly stable and reliable. The variable capacitor then becomes part of, for example, an oscillator circuit whose frequency is proportional to the force of the contact end 6 on the patient's cornea.

### MAGNETIC

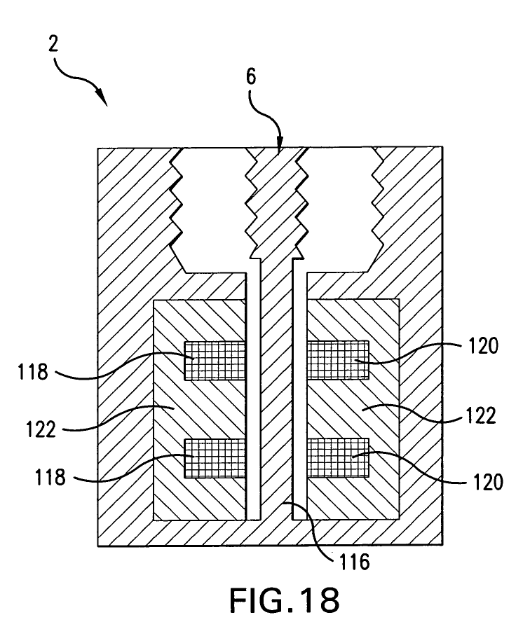

MEMS device 10 may also be a magnetic device. Such a MEMS device 10 is shown in FIG. 18. The MEMS device 10 of FIG. 18 forms a magnetic circuit wherein the contact end 6 is connected to a spring member 116. Application of a force to the contact end 6 causes mechanical deflection of spring member 116 as a function of the force. The MEMS device 10 of this embodiment includes a spring member 116 made of a magnetic, high-permeability material. Spring member 116 is centrally located between two coils 118 and 120 made of insulated wire. The coils 118, 120 are surrounded by and magnetically isolated from each other by insulating barriers such as nonmagnetic welded stainless steel barriers 122. Coils 118, 120 are electrically connected as part of an oscillator circuit. As the inductance of coils 118, 120 changes due to movement of contact end 6, the oscillation frequency of the oscillator circuit changes.

The electrical configuration of this MEMS device 10 is that of an inductive half-bridge. This half-bridge is driven by an alternating voltage source in the range typically of 1 KHz to 10 KHz. The centrally-disposed spring member 116 results in an inductive push-pull arrangement where deflection of the spring member 116 reduces the inductance of one coil (e.g. coil 118) and increases the inductance of the other (i.e. coil 120) creating a difference in coil impedance. The variation in the magnetic reluctance produces the effective inductance modulation as a function of the parameter input, in this case, the force applied to the contact end 6 by the patient's cornea.

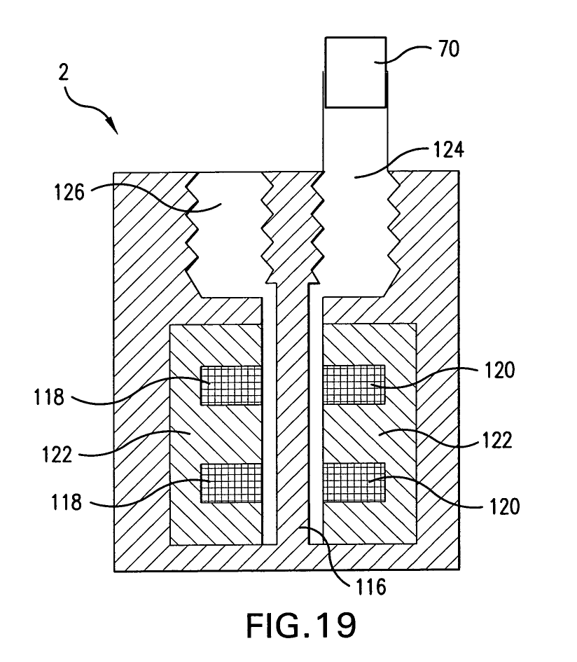

A variant of the embodiment of FIG. 18 is shown in FIG. 19. In this embodiment MEMS device 10 includes a first chamber 124 and a second chamber 126 formed on either side of spring member 116. Also, contact end 6 is not attached directly to spring arm 116. Instead, contact end 116 is attached to a piston 70 that is placed in first chamber 124 in a fluid-tight manner. Second chamber 126 is exposed to either a fixed or ambient pressure. This fixed or ambient pressure becomes the reference pressure for the MEMS device 10 in this embodiment. First chamber 124 is exposed to pressure created by the contact end 6 moving piston 70 in response to force applied to contact end 6 as the contact tonometer 2 is moved into contact with the patient's cornea. As can be seen, as the contact end 6 is moved into contact with the patient's cornea, a pressure difference will result between first chamber 124 and second chamber 126. This pressure differential will cause the spring member 116 to deflect from its resting position to a position in response to the differential pressure. Specifically, the increase in pressure in first chamber 124 will cause a difference in pressure between the first chamber 124 and the second chamber 126 that will move the spring member 116 towards the coil 118. This will result in modulation of the inductance (L) of the two coils 118, 120 which will be correlated by suitable electronics 12 to indicate the patient's IOP.

The following documents/products are incorporated herein by reference to provide exemplary disclosures of MEMS technology for use with the present invention: English, J. M. et. al., “Wireless Micromachines Ceramic Pressure Sensors,” IEEE, 1999, pp. 511-516; U.S. Patent Application Publication No. 2002/0121135 A1 to Rediniotis et. al; Kulsite XCS-062 differential pressure transducer using a fully active Wheatstone bridge on a silicone membrane; S. Sugiyama et. al. “Micro-diaphragm Pressure Sensor,” IEEE Int. Electron Devices Meetings, 2986, pp. 184-7, and H. Tanigawa et. al; “MOS Integrated Silicon Pressure Sensor,” IEEE Trans Electron Devices, Vol. ED-32, No. 7, pp. 1191—Jul. 15, 1985; U.S. Patent Application Publication No. 2002/0115920 A1 to Rish et al; U.S. Patent Application Publication No. 2002/0073783 A1 to Wilner et al; U.S. Patent Application Publication No. 2002/0049394 A1 ro Roy et al; U.S. Patent Application Publication No. 2002/0045921 A1 to Wolinsky et al; U.S. Patent Application Publication No. 2002/0029814 A1 to Unger et al; U.S. Patent Application Publication No. 2002/0029639 A1 to Wagner et al; U.S. Pat. No. 6,408,878 to Under et al; U.S. Pat. No. 6,367,333 B1 to Bullister et al; U.S. Pat. No. 6,341,528 B1 to Hoffman et al; U.S. Pat. No. 6,183,097 B1 to Scref et al; U.S. Pat. No. 6,460,234 B1 to Gianchandani; and U.S. Pat. No. 6,188,477 B1 to Pu et al. The MEMS transducer/sensor technology can sense pressure based on capacitive, electrostriction, magnetic, electromagnetic, thermoelastic, piezoelectric, piezoresistive, optical, resonance or other suitable effects.

The contact tonometer 2 of the present invention has been described herein as a handheld device. However, it is also within the scope of the invention for the contact tonometer 2 to be in a desktop or benchtop form. In such embodiments, the housing 4 would be attached to or include a base that rests on the desk or bench. In this embodiment the contact end 6 would be presented to contact a patient's cornea by mechanically moving the contact end 6 into such contact. Such means for moving the contact end are well within the scope of normal mechanical engineering so are not presented in detail at this time.

Inasmuch as the present invention is subject to various modifications and changes in detail that will be clear to those skilled in the art, it is intended that all subject matter discussed above and shown in the accompanying drawings be used as examples of the present invention and therefore should not be taken in a limiting sense. It is clear that changes and modifications to the description given herein including the drawings can be made and still be within the scope of the invention.

1. A contact tonometer for sensing intra-ocular pressure (IOP) of an eye comprising:
(a) a contact surface for making contact with a surface of said eye;
(b) a micro-electro-mechanical system (MEMS) device connected to said contact surface wherein said MEMS device produces an electrical signal corresponding to the force applied by said contact surface to said surface of said eye when said surface of said eye is contacted by said contact surface;
(c) an electronics unit for receiving said electrical signal and converting said electrical signal to an IOP signal that is representative of the IOP of the eye;
(d) a display for receiving the IOP signal from the electronics unit and displaying information that is representative of the IOP of the eye; and
(e) a power source for supplying electrical power to said electronics unit and said display.

2. The contact tonometer of claim 1 further comprising an activation switch connected to said power source.

3. The contact tonometer of claim 1 further comprising a membrane disposed at the contact surface and positioned between the contact surface and the surface of the eye.

4. The contact tonometer of claim 3 wherein the membrane is non-reactive and bio-compatible with the surface of the eye.

5. The contact tonometer of claim 4 wherein the membrane is disposable.

6. The contact tonometer of claim 1 wherein the power source is comprised of batteries.

7. The contact tonometer of claim 1 wherein the power source is comprised of common household electrical power provided through a power line.

8. The contact tonometer of claim 1 wherein the MEMS device and the electronics unit are formed together in an integrated circuit.

9. The contact tonometer of claim 1 wherein the MEMS device, the display and the electronics unit are formed together in an integrated circuit.

10. The contact tonometer of claim 1 wherein the electronics unit comprises a microprocessor.

11. The contact tonometer of claim 1 wherein the electronics unit comprises an application specific integrated circuit.

12. The contact tonometer of claim 1 wherein the MEMS device is in direct contact with the contact surface.

13. The contact tonometer of claim 1 further comprising a first housing member capable of being attached to a human finger for containing the contact surface and the MEMS device.

14. The contact tonometer of claim 13 further comprising an activation switch connected to said power source.

15. The contact tonometer of claim 13 further comprising a membrane disposed at the contact surface and positioned between the contact surface and the surface of the eye.

16. The contact tonometer of claim 15 wherein the membrane is non-reactive and bio-compatible with the surface of the eye.

17. The contact tonometer of claim 16 wherein the membrane is disposable.

18. The contact tonometer of claim 13 wherein the power source is comprised of batteries.

19. The contact tonometer of claim 13 wherein the power source is comprised of common household electrical power provided through a power line.

20. The contact tonometer of claim 13 wherein the first housing member further contains the electronics unit.

21. The contact tonometer of claim 20 wherein the MEMS device and the electronics unit are formed together in an integrated circuit.

22. The contact tonometer of claim 13 wherein the electronics unit comprises a microprocessor.

23. The contact tonometer of claim 13 wherein the electronics unit comprises an application specific integrated circuit.

24. The contact tonometer of claim 13 wherein the MEMS device is in direct contact with the contact surface.

25. The contact tonometer of claim 13 further comprising a second housing member coupled to said first housing member and capable of being attached to a human hand for containing the display.

26. A hand-held contact tonometer for sensing intra-ocular pressure (IOP) of an eye comprising:
(a) a contact surface for making contact with a surface of said eye;
(b) a micro-electro-mechanical system (MEMS) device connected to said contact surface wherein said MEMS device produces an electrical signal corresponding to the force applied by said contact surface to said surface of said eye when said surface of said eye is contacted by said contact surface;
(c) an electronics unit for receiving said electrical signal and converting said electrical signal to an IOP signal that is representative of the IOP of the eye;
(d) a display for receiving the IOP signal from the electronics unit and displaying information that is representative of the IOP of the eye; and
(e) a power source for supplying electrical power to said electronics unit and said display.

27. The hand-held contact tonometer of claim 26 further comprising a housing member capable of being hand-held for containing the contact surface, the MEMS device, the electronics unit and the display.

28. The hand-held contact tonometer of claim 27 further comprising a membrane disposed at the contact surface and positioned between the contact surface and the surface of the eye.

29. The hand-held contact tonometer of claim 28 wherein the membrane is non-reactive and bio-compatible with the surface of the eye.

30. The hand-held contact tonometer of claim 29 wherein the membrane is disposable.

31. The hand-held contact tonometer of claim 27 wherein the power source is comprised of common household electrical power provided through a power line.

32. The hand-held contact tonometer of claim 27 wherein the MEMS device and the electronics unit are formed together in an integrated circuit.

33. The hand-held contact tonometer of claim 27 wherein the MEMS device, the display and the electronics unit are formed together in an integrated circuit.

34. The hand-held contact tonometer of claim 27 wherein the electronics unit comprises a microprocessor.

35. The hand-held contact tonometer of claim 27 wherein the electronics unit comprises an application specific integrated circuit.

36. The hand-held contact tonometer of claim 27 wherein the MEMS device is in direct contact with the contact surface.

37. The hand-held contact tonometer of claim 27 wherein the power source is comprised of batteries.

38. The hand-held contact tonometer of claim 37 wherein the housing further contains the power source.
